(19) United States
(12) Patent Application Publication
(10) Pub. No.: US 2010/0262025 A1
(43) Pub. Date: Oct. 14, 2010

(54) APPARATUS FOR MEASUREMENT OF HEART RATE VARIABILITY

(75) Inventors: WEI-CHIH HU, CHUNG LI (TW); CHAO-FENG CHANG, CHUNG LI (TW)

Correspondence Address: ROSENBERG, KLEIN & LEE, 3458 ELLICOTT CENTER DRIVE-SUITE 101, ELLICOTT CITY, MD, 21043, US

(73) Assignee: CHUNG YUAN CHRISTIAN UNIVERSITY, CHUNG LI (TW)

(21) Appl. No.: 12/727,560

(22) Filed: Mar. 19, 2010

(30) Foreign Application Priority Data
Apr. 9, 2009 (TW) 098111871

(51) Int. Cl. A61B 5/02

(52) U.S. Cl. 600/504

(57) ABSTRACT

The present invention provides an apparatus for measurement of heart rate variability, comprising a main body of a earphone to be inserted into a patient's ear, a light source disposed on one side of the main body, emitting light projected onto the skin of the patient's ear, and a sensor disposed on the same side of the main body with the light source, receiving a reflection light from the skin of the patient's ear, and accordingly generating and transmitting a sensing signal to an analyzer for measurement of the heart rate variability. Thus, the heart rate variability of the patient can be measured by operation of the light source and the sensor when the patient is listening to the music through the earphone. In this way, true heart rate variability, without being affected by the patient's nervousness or impatience, can be derived.

### BACKGROUND OF THE INVENTION

1. Field of the Invention

The present invention is related to a measurement, more particularly, to a measurement of heart rate.

2. Description of the Prior Art

Heart rate variability (HRV) is a measure of the beat-to-beat variations in heart rate (beat per minute, BPM) and was proposed by Akselrod in 1981. It is a non-invasive electrocardiographic method wherein a power spectrum of the heart rate is obtained through Fourier Transform. Simply speaking, HRV refers to the differences among beat-to-beat intervals, i.e., the variations in the frequency of heartbeat and the lengths of the beat-to-to intervals. The power spectrum reflects autonomic nervous system activities. Autonomic dysfunction or some kinds of heart disease can be detected by measurement of HRV.

The autonomic nervous system (ANS or visceral nervous system) is the part of the peripheral nervous system that acts as a control system functioning largely below the level of consciousness, and controls visceral functions. It can be divided by subsystems into the parasympathetic nervous system and sympathetic nervous system. The sympathetic nervous system becomes more active during times of stress and is responsible for priming the body for action. The sympathetic nervous system activity will increase energy expenditure. The parasympathetic nervous system is active during relaxation and rest. Activity in the parasympathetic nervous system decreases heart rate and blood pressure and promotes digestion, and thus has the effect of energy storage.

Heart rate variability is a powerful measure of autonomic nervous system function. Since HRV is a marker of sympathetic and parasympathetic influences on the modulation of heart rate, spectral analysis of HRV has been applied in many medical researches to study autonomic nervous system activity. Autonomic imbalance, as well as some kinds of heart disease, can be detected by measurement of HRV. A patient having a reduced HRV suffers from a high risk of heart disease. HRV plays an important role in early diagnosis or prediction of numerous diseases, such as arrhythmia, diabetes and melancholia.

An arrhythmia is a problem with the speed or rhythm of the heartbeat. During an arrhythmia, the heart can beat too fast, too slow, or with an irregular rhythm. A heartbeat that is too fast is called tachycardia while a heartbeat that is too slow is called bradycardia. Most arrhythmias are harmless, but some can be serious or even life threatening. Harmless arrhythmias include mild acceleration and slowing of the heart rate that occurs with breathing in and out, or during exercise and sleep. Other events such as autonomic nervous system activity, drinking coffee or tea, fever, nervousness, stress, pain, anoxia and drugs also affect heart rate.

Some arrhythmias are life-threatening medical emergencies that can result in cardiac arrest and sudden death. Others cause symptoms such as an abnormal awareness of heart beat (palpitations), and may be merely annoying. Still others may not be associated with any symptoms at all, but predispose toward potentially life-threatening stroke or embolus. Reduced heart rate variability is valuable in predicting numerous diseases. The risk of sudden death can be lowered by countinuous HRV monitoring.

HRV has a special interest for early detection of autonomic neuropathy in diabetic patients. At early stage of diabetes, the patient will have a normal blood sugar level but a reduced HRV. Diabetic patients at intermediate or final stage will suffer from autonomic neuropathy. Nerve disorders caused by autonomic neuropathy will result in symptoms such as dizziness, palpitations, night sweat and diarrhea. HRV results of diabetic patients significantly deviates from their normal HRV and therefore can be used to validate the effect of therapy they received.

Depression is a serious medical illness that involves the brain. It's more than just a feeling of being “down in the dumps” or “blue” for a few days. More than 20 million people in the United States have depression. Women are diagnosed with depression twice as often as men. People with heart disease, apoplexy, cancer and diabetes are more likely to have depression. Reduced HRV is common in depression patients. Many medical literatures demonstrate the validity of numerous prescription drugs on depression. Statistically, there are 80˜90% of patients recovering from depression by receiving psychotherapy or drug treatment. HRV can be a tool for validation and monitoring of the effect of the therapy or treatment.

HRV is assessed from calculation of the mean R-R interval and its standard deviation measured on electrocardiograms for a duration of about ten minutes or more. The first step is re-sampling of the R-R interval. A power spectrum of the re-sampled data is then derived by Fourier Transform. The frequency components of the power spectrum within a higher frequency band (0.15˜0.4 Hz) and a lower frequency band (0.04˜0.15 Hz) are used to assess the autonomic nervous system activity.

During the measurement of HRV on electrocardiograms, the patients would be nervous or impatient if they pay too much attention to the measurement for such a relatively long time. This can affect the HRV results. However, HRV results derived from a short-term measurement is helpless to diagnosis of heart diseases such as occasional arrhythmia.

Therefore, the present invention provides an apparatus for measurement of heart rate variability, wherein the true heart rate variability, without being affected by the patient's nervousness or impatience, can be derived.

### SUMMARY OF THE INVENTION

The present invention provides an apparatus for measurement of heart rate variability which is characterized by an earphone having a light source and a sensor disposed thereon. The heart rate variability of the patient can be measured by operation of the light source and the sensor when the patient is listening to the music through the earphone. This prevents patients from paying too much attention to the measurement, whereby the true heart rate variability, without being affected by the patient's nervousness or impatience, can be derived.

The present invention provides an apparatus for measurement of heart rate variability, comprising a main body of a earphone to be inserted into a patient's ear, a light source disposed on one side of the main body, emitting light projected onto the skin of the patient's ear, and a sensor disposed on the same side of the main body with the light source, receiving a reflection light from the skin of the patient's ear, and accordingly generating and transmitting a sensing signal to an analyzer for measurement of the heart rate variability. Thus, the heart rate variability of the patient can be measured by operation of the light source and the sensor when the patient is listening to the music through the earphone. In this way, true heart rate variability, without being affected by the patient's nervousness or impatience, can be derived.

### BRIEF DESCRIPTION OF DRAWINGS

FIG. 1 is a diagram showing the apparatus for measurement of heart rate variability alone according to one embodiment of the invention.

FIG. 2 is a diagram showing the apparatus for measurement of heart rate variability being inserted into a patient's ear according to one embodiment of the invention.

FIG. 3 shows a PPG result.

FIGS. 4A and 4B show the structure of an apparatus for measurement of heart rate variability according to another embodiment of the invention.

### DETAILED DESCRIPTION OF PREFERRED EMBODIMENTS

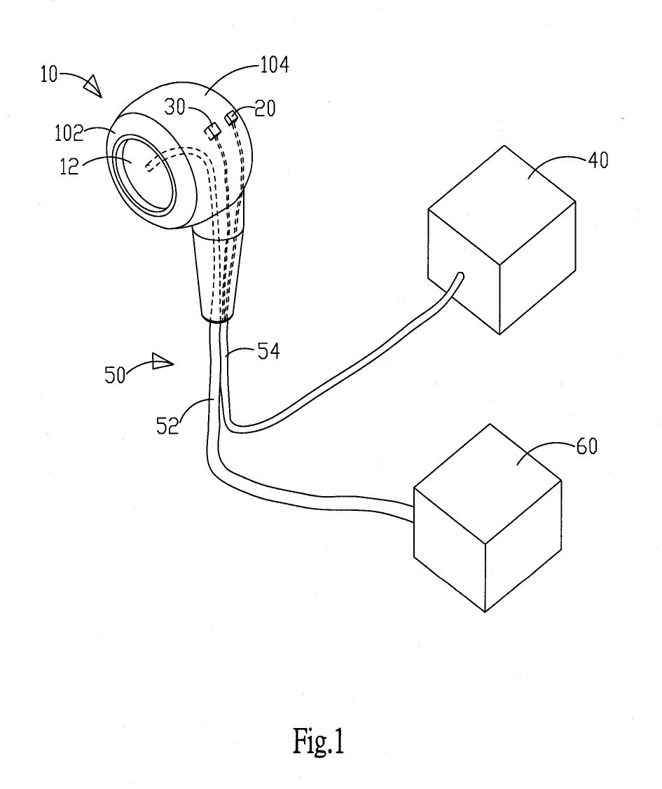

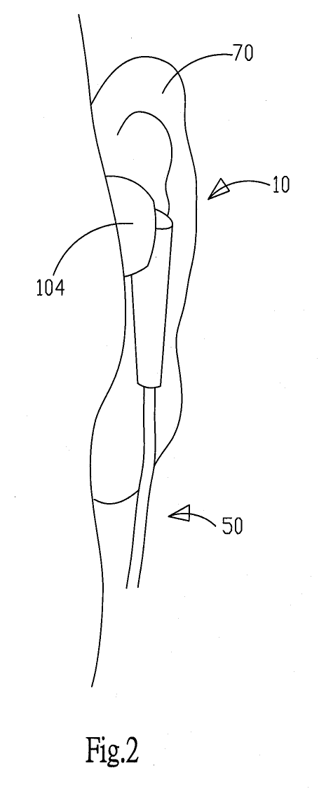

FIG. 1 and FIG. 2 are diagrams showing the apparatus for measurement of heart rate variability alone and when being inserted into a patient's ear according to one embodiment of the invention. The apparatus includes a main body 10, a light source 20 and a sensor 30. The main body 10 is to be inserted into the patient's ear and includes a speaker 12. The light source 20 is disposed on one side of the main body 10 for emitting light projected onto the skin of the patient's ear. The sensor 30 is disposed on the same side of the light source 20 on the main body 10 and receives a reflection light from the skin of the patient's ear. A sensing signal is transmitted by the sensor 30 to an analyzer 40 for measurement of heart rate variability.

The main body 10 includes an insertion portion 102 and a container portion 104. The insertion portion 102 is to be inserted into the patient's ear 70. The speaker 12 is disposed in the insertion portion 102. The light source 20 and sensor 30 are disposed on the container portion 104. By listening to the music through the earphone (main body), the patient does not pay much attention to the measurement. This avoids the patient's nervousness or impatience during measurement and makes the measurement result unaffected.

In the previously described apparatus, the measurement is performed by PPG (Photoplethy-smography). It acquires a probe composed of the light source 20 implemented by a red light emitting diode and a sensor 30 implemented by a photo transistor. The wave length of the red light emitting from the light source 20 is 640 nm. Since the sensor 30 implemented by the photo transistor is too small to be felt, the patient is not conscious of the existing of the PPG probe on the earphone (main body) by which he (she) is listening to the music. This also avoids the patient's nervousness or impatience during measurement and makes the measurement result unaffected.

Additionally, the previously described apparatus further includes a transmission line set 50 coupled to the speaker 12, light source 20 and sensor 30. The transmission line set 50 includes an audio signal line 52 coupled to the speaker 12 and a sensing signal line 54 coupled to the light source 20, sensor 30 and the analyzer 40. A digital music player 60 such as CD player or MP3 player may be connected to the audio signal line 52. The light source 20 is controlled by the signal on the sensing signal line 54 while the analyzer 40 receives the sensing signal from the sensor 30 through the sensing signal line 54.

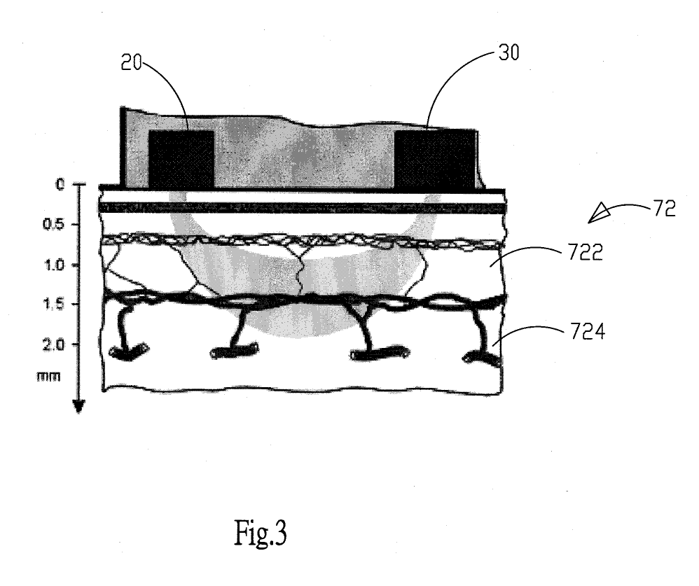

FIG. 3 shows a PPG result. A PPG is often obtained by using an near infrared light source 20 illuminating a selected area of the skin and measures changes in light absorption. When light propagates into a human body, it will be absorbed in many ways, such as being absorbed by skin, bones or blood in the artery or vein. When the heart pumps blood to the periphery, the artereis and arterioles are distended due to this pressure pulse, which changes the absorption of light. This change caused by the pressure pulse is detected by illuminating the skin with the light from a light source and then measuring the amount of reflection light from the skin. The measuring result includes an AC and DC component. The magnitude of the AC component is typically 1%˜2% of that of the DC component. In the embodiment shown in FIGS. 1 and 2, the light source 20 emits light penetrating the epidermis 722 of the skin 72 of the patient's ear 70 and reaching the dermis 724 underneath the epidermis 722, which results in a reflection light from the skin 72. The sensor 30 receives the reflection light and accordingly generates a sensing signal transmitted to the analyzer 40 for measurement of heart rate variability.

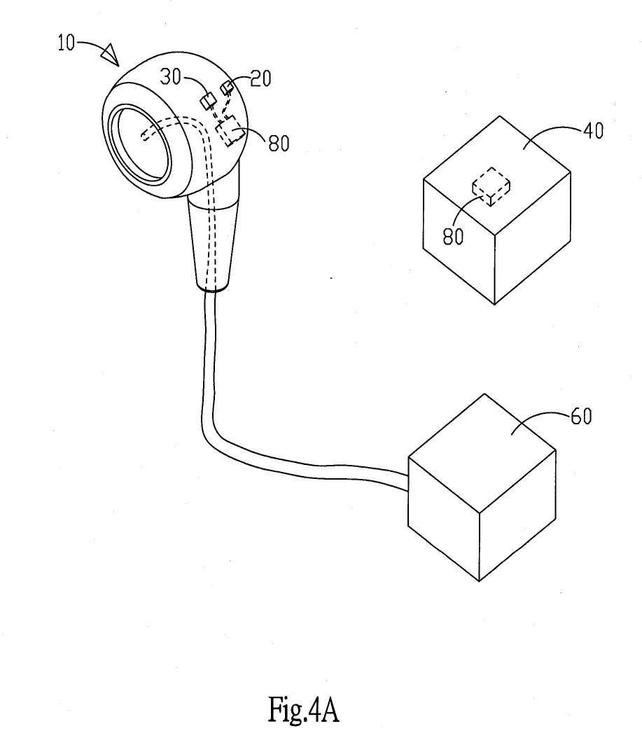

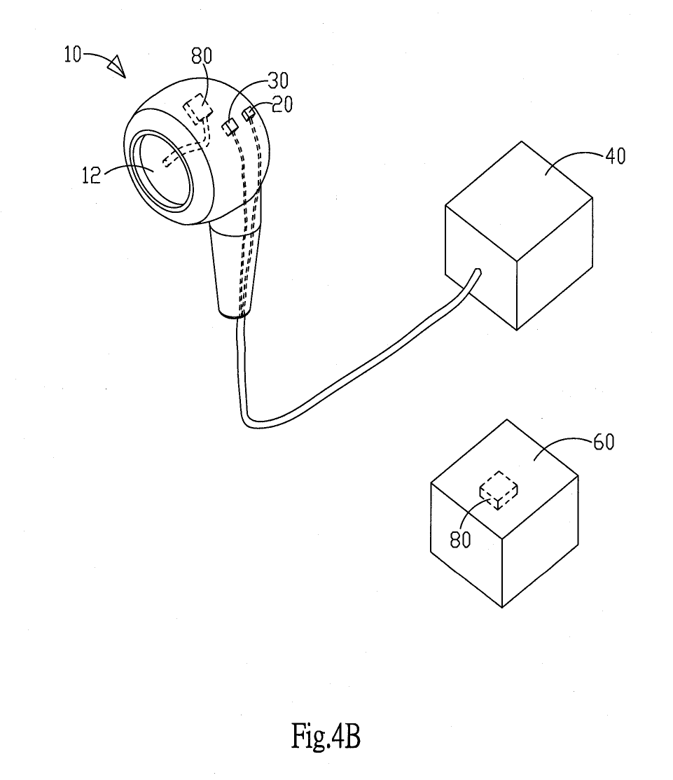

FIGS. 4A and 4B show the structure of an apparatus for measurement of heart rate variability according to another embodiment of the invention. The apparatus of FIGS. 4A or 4B is different from that of FIGS. 1 or 2 in the signal transmission. The signal transmission is carried out through the transmission line set 50 in the apparatus of FIGS. 1 or 2, while the apparatus of FIGS. 4A or 4B includes two wireless tranceiver modules 80 wherein one is disposed in the main body 10 and the other in the analyzer 40. The sensor 30 and speaker 12 are coupled to the wireless transceiver module 80 in the main body 10 respectively for transmission of the sensing signal to the analyzer 40 and receiving of the audio signal from a digital player 60 having another corresponding wireless transceiver module 80.

In conclusion, the present invention provides an apparatus for measurement of heart rate variability, comprising a main body of a earphone to be inserted into a patient's ear, a light source disposed on one side of the main body, emitting light projected onto the skin of the patient's ear, and a sensor disposed on the same side of the main body with the light source, receiving a reflection light from the skin of the patient's ear, and accordingly generating and transmitting a sensing signal to an analyzer for measurement of the heart rate variability. Thus, the heart rate variability of the patient can be measured by operation of the light source and the sensor when the patient is listening to the music through the earphone. In this way, true heart rate variability, without being affected by the patient's nervousness or impatience, can be derived.

What is claimed is:

1. An apparatus for measurement of heart rate variability, comprising:
a main body of a earphone to be inserted into a patient's ear;
a light source disposed on one side of the main body, emitting light projected onto the skin of the patient's ear; and
a sensor disposed on the same side of the main body with the light source, receiving a reflection light from the skin of the patient's ear, and accordingly generating and transmitting a sensing signal to an analyzer for measurement of the heart rate variability.

2. The apparatus as claimed in claim 1, wherein the light source emits red light.

3. The apparatus as claimed in claim 2, wherein the wave length of the red light is 640 nm.

4. The apparatus as claimed in claim 1, wherein the light source is a red light emitting diode.

5. The apparatus as claimed in claim 1, wherein the sensor comprises a photo transistor.

6. The apparatus as claimed in claim 1, wherein the light emitted from the light source penetrates the epidermis and reaches the dermis of the skin of the patient's ear, and the reflection light is reflected from the dermis.

7. The apparatus as claimed in claim 1, wherein the main body comprises a speaker.

8. The apparatus as claimed in claim 7, wherein the main body comprises:
an insertion portion to be inserted into the patient's ear, in which the speaker is disposed;
a container portion on which the light source and the sensor are disposed.

9. The apparatus as claimed in claim 8 further comprising transmission lines coupled to the speaker, the light source and the sensor.

10. The apparatus as claimed in claim 9, wherein the transmission lines comprise:
an audio signal line coupled to the speaker; and
a sensing signal line coupled to the light source, the sensor and the analyzer.

11. The apparatus as claimed in claim 8 further comprising a wireless transceiver module transmitting an audio signal to the speaker.

12. The apparatus as claimed in claim 1 further comprising a wireless transceiver module transmitting the sensing signal to the analyzer.
